(19) United States
(12) Patent Application Publication
(10) Pub. No.: US 2004/0121489 A1
(43) Pub. Date: Jun. 24, 2004

(54) COX-2 mediated altered prostaglandin balance in diabetes complications

(76) Inventors: Vikas R. Dharnidharka, Gainesville, FL (US); Michael J. Clare-Salzler, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/652,216

(22) Filed: Aug. 29, 2003

Related U.S. Application Data
(60) Provisional application No. 60/407,150, filed on Aug. 29, 2002.
(60) Provisional application No. 60/465,016, filed on Apr. 23, 2003.

(51) Int. Cl. G01N 33/543

(52) U.S. Cl. 436/518

(57) ABSTRACT

The subject invention concerns novel methods for determining an individual's risk of developing diabetes-related complications. An embodiment provides determining at the onset or prior to the onset of Type I diabetes those individuals with a high risk of developing diabetes-related complications. The subject invention also concerns therapeutic methods for inhibiting and/or preventing the development of diabetes-related complications prior to or at the onset of T1D.

### CROSS-REFERENCE TO RELATED APPLICATION

This application claims the benefit of U.S. Provisional Application No. 60/407,150, filed Aug. 29, 2002 and U.S. Provisional Application No. 60/465,016, filed Apr. 23, 2003.

### BACKGROUND OF INVENTION

Diabetes is a term that refers to a collection of diseases resulting in disordered energy metabolism and varying degrees of blood glucose elevations or hyperglycemia. One of the best characterized forms of the disease is that which arises from an immunologically mediated destruction of the insulin secreting pancreatic beta cells. This severe form of the disease is termed Insulin-Dependent Diabetes (IDD or IDM) since it is associated with progressive insulin deficiency and coincident symptoms such as weight loss, glycosuria and polyuria, and increased thirst or polydipsia. Other terms for this form of diabetes are Type 1 Diabetes (T1D, cf. Type 2 Diabetes which results from an inherent resistance to insulin action); Ketosis Prone Diabetes because there is abnormal generation of ketone bodies as a result of excessive breakdown of body fats due to the severe insulin deficiency; or Juvenile Diabetes, since virtually all diabetes that appears in childhood and adolescence are of this type.

Diabetes is a major public health problem, especially in Western countries. The incidence rates vary greatly worldwide, from as high as 40 per 100,000 persons in Finland to as low as 1-2 per 100,000 among the Japanese. The peak incidence is during the pubertal years, associated with the increasing bodily demands for insulin associated with muscle growth. The prevalence rates in the United States population under age 20 years is 0.25% and it approaches 0.4% over a lifetime, albeit an estimated 10-20% of patients with Non Insulin-dependent Diabetes (NIDD) or Type 2 or Maturity Onset Diabetes also have, in reality, slowly progressive IDD. Thus, it is estimated that there may be at least 1 million Americans affected by IDD.

There are namely four serious complications of diabetes: diabetic nephropathy or kidney disease; diabetic retinopathy which causes blindness due to destruction of the retina; diabetic neuropathy involving the loss of peripheral nerve function; and arteriosclerosis as well as other circulatory problems due to capillary damage. These complications are a significant concern to society as well as diabetic individuals. Ocular complications of diabetes are the leading cause of new blindness in persons 20-74 years of age. Approximately 40% of individuals with T1D develop severe nephropathy and kidney failure by the age of 50, with some developing kidney failure before the age of 30. Additionally, patients with T1D are prone to other diseases including, for example, thyroiditis or Hashimoto's disease, Graves' disease, Addison's disease, atrophic gastritis and pernicious anemia, celiac disease, and vitiligo (Maclaren, N. K. [1985] Diabetes Care 8(suppl.):34-38).

Various factors have been considered to be responsible for the onset and progression of complications of diabetes. For example, both retinopathy and nephropathy are conditions believed to be related to general circulatory problems often associated with diabetes. Of all patients with diabetes, those with T1D have a disproportionate share of the complications described above due to the severity and early age of onset of the disease. Diabetes is one of the most costly health problems in the United States. In 1997, it was estimated that $54 billion in economic cost was attributed to indirect health care costs in diabetes-related disability and mortality.

As described above, complications from diabetes such as nephropathy, retinopathy and atherosclerosis lead to kidney failure, blindness and heart disease, respectively. These complications are associated with a high degree of suffering, increased mortality rates and enormous medical costs. Because symptoms related to these complications usually occur only in the late stages of diabetes, when biological function has decreased considerably to less than normal capacity, detection of background cellular factors expressed early in the disease process is of great clinical importance. In particular, detection of these factors would preferably identify individuals before the occurrence of any diabetes-related complications. Early identification of populations of patients at high-risk for complication and the ability to prevent such complications would result in better patient health, reduced human suffering, and a large reduction in health care costs associated with complications related to Type 1 Diabetes (T1D). Thus it is important that the pathogenesis of diabetes-related complications be understood and strategies be developed to prevent the onset of these complications.

The metabolites of arachidonic acid, such as prostaglandins, lipoxygenases, and thromboxane products are produced throughout the body in multiple tissues and perform diverse biological functions. Prostaglandins mediate both beneficial and undesirable biological reactions. They regulate blood flow and salt-water balance in the kidney and are also important in platelet aggregation. Recent studies suggest that prostaglandins may play an important role in detecting diabetes. For example, it has been demonstrated that aberrant expression of an enzyme in prostaglandin production is a feature of diabetes susceptibility (Leslie et al., [2001] Diabetes, 50(suppl.): A198).

Prostaglandins (PGs) are produced from cell membrane phospholipids by a cascade of enzymes. These enzymatic activities involve the conversion of arachidonic acid to a common prostaglandin precursor, PGH₂, by specific enzymes called prostaglandin synthases (cyclooxygenase-1 and cyclooxygenase-2). PGH₂ is eventually converted to various types of prostaglandins (also known as prostanoids) including, for example, PGE₁, PGE₂, or prostacyclin, PGF_{2α} and thromboxanes, by cell-specific synthases.

The enzyme cyclooxygenase-1 (also known as COX-1, prostaglandin G/H synthase I, PGS-1) is constitutively expressed, responsible for the production of low levels of PGs, and functions as a housekeeping molecule. Cyclooxygenase-2 (also known as COX-2, prostaglandin G/H synthase 2, PGS-2) is an inducible enzyme expressed by macrophages and monocytes during inflammation and following exposure to mitogens, cytokines, and bacterial cell wall products, i.e., lipopolysaccharide (LPS) (Farber, J. M. [1992] Mol. Cell. Biol. 12:1535-1545; Vane, J. R. [1994] Proc. Natl. Acad. Sci. USA 91:2046-2050; Kujubn, D. A. [1993] J. Biol. Chem. 266:12866-12872; Ristimaki, A. et al. [1994] J. Biol. Chem. 269:11769-11775). COX-2 has been shown to be expressed in the cells lining the joints of individuals with rheumatoid arthritis and may contribute to the ongoing inflammation in the affected joint (Crofford, L. J. et al. [1994] J. Clin. Invest. 93:1095-1101).

The mechanisms underlying diabetes-related complications are complex and multi-factorial. Not all diabetic individuals, including T1D individuals, develop the complications described above and there are no predictive markers for these complications at the onset of IDM. Currently, individuals can be screened only after the onset of diabetes for the probability of their developing a diabetes-associated pathologic condition. This screen measures fructose lysine, 3-deoxyglucosone, and 3-deoxyfructose levels in urine samples before and after ingestion of a source of glycated lysine. Unfortunately, because the onset of diabetic pathologic conditions can be gradual and without symptoms, diabetic individuals who are eventually screened may already be afflicted with a condition. No test exists, immunologic, genetic, or otherwise, that can identify individuals at risk for diabetic complications in order to prevent or delay the development of these complications. In particular, there are no tests at the time of onset of diabetes or prior to the onset of diabetes that can predict who is at higher risk for subsequent kidney, nerve, vasculature, or retinal damage.

### BRIEF SUMMARY

The present invention relates to materials and methods for the detection, prevention, and treatment of diabetes-related complications. In particular, the present invention arose from the discovery that prostanoids play a role in diabetes-related complications.

In a specific embodiment, the subject invention concerns the identification of an imbalance in prostanoid levels in order to identify individuals who are developing, or who are at risk for developing complications associated with T1D, including in individuals with pre-T1D. Pre-T1D individuals are individuals that maintain normal glucose levels that are predisposed to developing T1D. These individuals can be identified using established methods including assays for autoantibodies specific for T1D and a loss of insulin section.

To assess the balance in prostanoid levels in an individual, net prostanoid activity is monitored. Net prostanoid activity is best reflected as a ratio of different prostanoids. In a preferred embodiment, the ratio of prostaglandin E to thromboxane B₂ is monitored to provide a profile on the individual's prostanoid levels. An imbalance in prostanoid levels (e.g., change in the individual's profile), provides an indication regarding the likelihood of diabetes-related complications occurring in the individual. A drop in net prostanoid activity can be a result of abnormal enzyme cyclooxygenase-2 activity.

One characteristic of prostanoid imbalance is a drop in the ratio value of renal prostanoid metabolites (i.e., ratio of prostaglandin E₂ to thromboxane B₂) in recent onset T1D and pre-T1D individuals as compared to net prostanoid activity in recent onset Type II diabetes mellitus and healthy age-matched individuals.

In one embodiment of the subject invention, individuals at risk for developing diabetes-related complications are identified on the basis of a drop in their ratio values of metabolites of renal prostanoids when compared to normal net prostanoid activity in individuals who have a lower risk of developing diabetes-related complications. In a specific embodiment of the subject invention, diabetes-related complications can be predicted by monitoring the ratio of the prostaglandin E₂ (PGE₂) to thromboxane B₂ (TXB₂).

Once a drop in net prostanoid activity (i.e., PGE₂/TXB₂) is verified as an indication of increased risk in developing diabetes-related complications, the present invention provides a method for restoring/increasing the net prostanoid activity in an individual. In one embodiment, cyclooxygenase-2 (COX-2) inhibitors are administered to the individual to increase net prostanoid activity. Administration of COX-2 inhibitors to increase net prostanoid activity can decrease or prevent the development of diabetes-related complications.

One aspect of the subject invention is the determination that there is a link between prostanoid imbalance and abnormally high renal filtration, which leads to the subsequent development of diabetic nephropathy. In a preferred embodiment, bodily fluid samples (i.e., urinary and blood samples) are analyzed for 24-hour prostanoid metabolites of PGE₂, PGI₂, TXA₂, 6 ketoPGF_{1α} (6kPGF_{1α}) and/or TXB₂ as well as systemic derived metabolites of bicyclo-PGE₂, 2,3 dinor 6 ketoPGF_{1α} and/or 11-dehydro-TXB₂.

The expression of prostanoids can be detected in any number of ways that would be apparent to those skilled in the art having the benefit of this disclosure. For example, the levels of PGE₂ and TXB₂ can be determined by using commercially available enzyme immunoassays such as BIOTRAK or using known standard protocols.

The diagnostic procedures described herein can be used to assess the risk of developing diabetes-related complications at the onset, or prior to the onset, of T1D. This early detection makes it possible to initiate appropriate preventative and remedial measures.

The use of prostanoid imbalance to identify individuals at a higher risk for developing diabetes-related complications such as retinal disease, nephropathy, and atherosclerosis, has found practical application in the present invention which, in one aspect, provides a method for preventing, reducing, or delaying the onset of diabetic complications in an individual at the onset of T1D or prior to the onset of T1D.

A further embodiment according to the present invention provides treatments for preventing or hindering the development of diabetes-related complications through the administration of one or more compounds in an amount effective to restore prostanoid balance or modulate eicosanoid production. In a preferred embodiment, selective COX-2 inhibitors are administered at the onset of, or prior to the onset of, T1D diabetes, to restore balance in net prostanoid activity.

### BRIEF DESCRIPTION OF DRAWINGS

FIG. 1 shows the paradigm for prostaglandin synthesis and their renal effects.

FIG. 2 shows that renal hemodynamic changes in pre-T1D and recent onset T1D individuals occur prior to the onset of hyperglycemia.

FIG. 3 shows the difference in urinary ratios for PGE₂, PGI₂ and TXB₂ (PGE₂/TXB₂ and PGE₂+6ketoPGF_{1α}/TXB₂) in pre-T1D and recent onset T1D individuals as compared to recent onset Type II diabetics and healthy age-matched controls.

FIGS. 4a and 4b show results in both non-obese diabetic (NOD) mice and in diabetes prone (DP) rats similar to those results demonstrated in FIG. 3.

FIG. 5 shows the absolute levels of prostanoid metabolites for systemic PGE₂ and TXA₂ in pre-T1D and recent onset T1D individuals as opposed to healthy age-matched controls or recent onset Type II diabetics.

FIG. 6 shows the ratio of urinary bicyclo PGE₂/11-dehydro TXB₂ levels in pre-T1D and recent onset T1D individuals when compared to controls and individuals with long-standing T1D.

FIG. 7 is a map of NOT C1t interval in B6.NODc1t strain mice.

FIG. 8 is a graphical illustration of the effect of COX-2 blockage on PGE₂/TXB₂ ratio.

FIGS. 9a and 9b are illustrations of COX-2 staining in kidneys of 6-8 week old female B6 and pre-diabetic NOD mice.

### DETAILED DISCLOSURE

In one aspect, the subject invention pertains to the detection and/or modulation of prostanoid balance in individuals prone to T1D or recently diagnosed with T1D. In a preferred embodiment, the subject invention concerns the discovery that prostanoids may contribute to differences in susceptibility or the rate of progression to diabetes-related complications including, for example, nephropathy, retinopathy, and atherosclerosis.

Another embodiment of the subject invention concerns preventative or therapeutic treatments. Drugs that restore prostanoid balance can be administered to individuals prone to T1D or recently diagnosed with T1D that have been identified as having a high risk for developing diabetes-related complications in the future.

As used herein, the term “T1D” refers to individuals with insulin-dependent diabetes mellitus (“Type I” diabetics, IDD, or IDM). The hyperglycemia present in individuals with Type I diabetes is associated with deficient, reduced, or nonexistent levels of insulin which are insufficient to maintain blood glucose levels within the physiological range. Common treatment of Type I diabetes involves administration of replacement doses of insulin, generally by a parental route.

As used herein, the term “pre-T1D” refers to individuals who are at a high risk for (or prone to) T1D before their disease becomes clinically apparent. These individuals often maintain normal glucose levels. Recently, tests have been developed to identify those individuals at risk for developing T1D. Immunological abnormalities of T1D (i.e., autoantibodies specific for T1D) are present in these high risk individuals before the clinical onset of hyperglycemia. Detection of these immunological abnormalities including, for example, autoantibodies directed against insulin, islet cell cytoplasm, and glutamic acid decarboxylase, may be used to determine an individual prone to T1D. Generally, roughly 60% of these pre-T1D subjects develop diabetes within 5 years.

As used herein, the term “diabetes-related complications” refers to diabetes-associated pathologic conditions including neuropathy, nephropathy, cardiomyopathy, myocardial infarction, ophthalmopathy, retinopathy, and atherosclerosis.

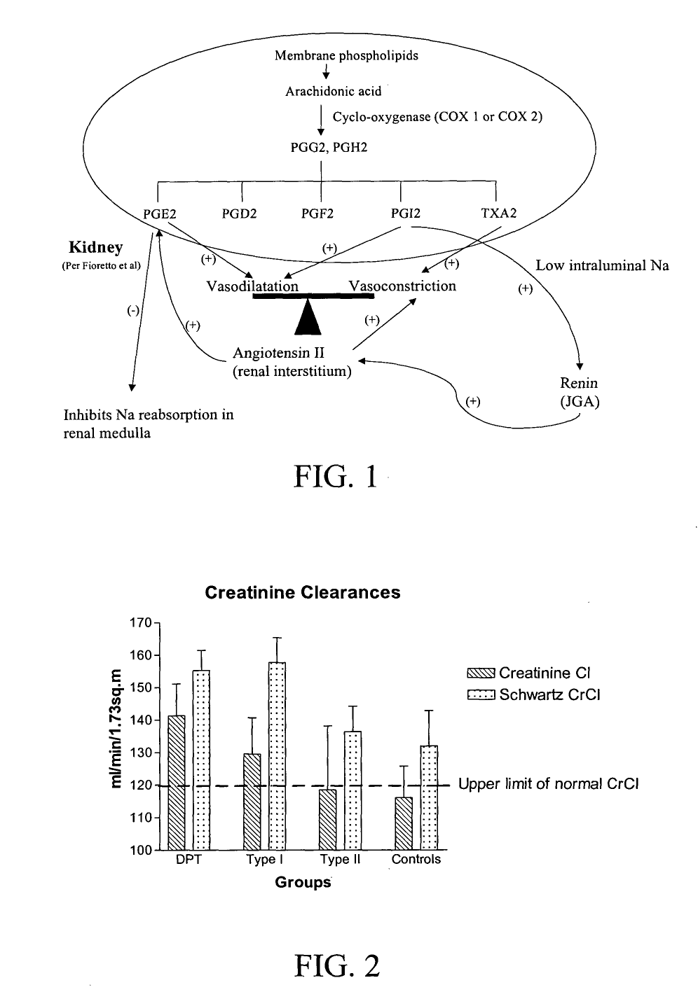

As used herein, the term “prostanoids” or “eicosanoids” refers to phospilolipidderived inflammatory mediators encompassing prostaglandins, prostacyclins and thromboxanes, as well as their respective metabolites. Fatty acids of the linoleic acid family (deriving from 18:2n-6) are the main source of eicosanoids, arachidonic acid (20:4n-6) being the major precursor. Eicosanoids are synthesized in vivo through several routes, with some compounds being formed by more than one mechanism. The main biosynthetic pathways of prostanoids are: cyclooxygenase, lipoxygenase and epoxygenase pathways. Prostanoids such as thromboxane are end products of prostaglandin metabolism. Prostanoids are produced throughout the body in multiple tissues and perform diverse functions. The major prostanoids involved in blood vessel regulation in different organs and in specific mesangial functions in the kidneys are prostaglandin E2 (PGE₂), prostacyclin (PGI₂) and thromboxane A2 (TXA₂). The levels of these substances in different body compartments can be measured by assaying the parent compound or their metabolites. PGE₂, 6ketoPGF1α, and TXB₂ are the compounds assayed for renal specific production of PGE₂, PGI₂ and TXA₂, respectively. As illustrated in FIG. 1, prostanoids can be produced via the enzymatic actions of COX on arachidonic acid in the kidney to modulate multiple renal functions including glomerular blood flow and mesangial function.

As used herein, the term “imbalance in prostanoid production” refers to the shift in balance (or ratio values) of prostanoids. According to the present invention, the imbalance in prostanoid production is characterized by a change in observed ratio values of prostanoids in an individual or a difference in ratio values of prostanoids in an individual as compared to normal net prostanoid activity in recent onset Type II diabetics and/or healthy age-matched controls. In a preferred embodiment, the imbalance in prostanoid production is characterized by a change in metabolites of renal prostanoids in pre-T1D individuals and recent onset T1D individuals as compared to normal net prostanoid activity in recent onset Type II diabetics and healthy age-matched controls. Irregular prostanoid production may be a result of abnormal regulation of the cyclooxygenase pathway.

The term “individual” or “patient,” as used herein, describes an animal, including mammals, to which methods in accordance with the present invemion are provideu. Mammalian species that benefit from the disclosed methods of the invention include, and are not limited to, humans, apes, chimpanzees, orangutans, monkeys; and domesticated animals such as mice, rats, guinea pigs, and hamsters.

The term “eicosanoid modulator,” as used herein, refers to agents which can modulate eicosanoid biological activity including, but not limited to, prostaglandins, leukotrienes and arachidonic acid, altered intermediates or enzymes of eicosanoid biosynthetic pathways, inhibitors of signal molecules which turn on eicosanoid biosynthesis, and, preferably, inhibitors of the eicosanoid biosynthetic pathway.

As used herein, the term cyclooxygenase-2 inhibitor or “COX-2 inhibitor” or “selective COX-2 inhibitor” defines an enzyme of eicosanoid biosynthetic pathways and shall include the following: all of the compounds and substances beginning on page 8 of Winokur WO99/20110 as members of three distinct structural classes of selective COX-2 inhibitor compounds, and the compounds and substances which are selective COX-2 inhibitors in Nichtberger, U.S. Pat. No. 6,136,804 and the compounds and substances which are selective COX-2 inhibitors in Isakson et al., PCT application WO/09641645 published Dec. 27, 1996, filed as PCT/US 9509905 on Jun. 12, 1995, entitled “Combination of a Cyclooxygenase-2 Inhibitor and a Leukotriene B4 Receptor Antagonist for the Treatment of Inflammations.” The meaning of COX-2 inhibitor in this invention shall include the compounds and substances referenced and incorporated into Winokur WO99/20110 by reference to art therein, the compounds and substances referenced and incorporated into Nichtberger, U.S. Pat. No. 6,136,804, Oct. 24, 2000, by reference to art therein, and the compounds and substances which are COX-2 inhibitors referenced and incorporated into Isakson et al, PCT application WO/09641645 published Dec. 27, 1996, filed as PCT/US 9509905 on Jun. 12, 1995, entitled “Combination of a Cyclooxygenase-2 Inhibitor and a Leukotriene B4 Receptor Antagonist for the Treatment of Inflammations.”

The meaning of COX-2 inhibitor in this invention also includes rofecoxib, and celecoxib, marketed as VIOXX and CELEBREX by Merck and Searle/Pfizer respectively. Rofecoxib is discussed in Winokur, WO99/20110 as compound 3, on p.9. Celecoxib is discussed as SC-58635 in the same reference, and in T. Penning, “Synthesis and biological evaluation of the 1,5-diarylpyrazole class of cyclooxygenase-2 inhibitors: identification of 4-[5-(4-methylphenyl)-3-(trifluoromethyl)-1H-pyrozol-1 yl]benzenesulfonami de (SC-58635, celecoxib),” J. Med. Chem. Apr. 25, 1997: 40(9): 1347-56. The meaning of COX-2 inhibitor in this invention also includes SC299 referred to as a fluorescent diaryloxazole. C. Lanzo et al, “Fluorescence quenching analysis of the association and dissociation of a diarylheterocycle to cyclooxygenasel-1 and cyclooxygenase-2: dynamic basis of cycloxygenase-2 selectivity,” Biochemistry May 23, 2000 vol. 39(20):6228-34, and in J. Talley et al, “4,5-Diaryloxazole inhibitors of cyclooxygenase-2 (COX-2),” Med. Res. Rev. May 1999; 19(3): 199-208. The meaning of COX-2 inhibitor in this invention also includes valdecoxib, see, “4-[5-Methyl-3-phenylisoxazol-1-yl]benzenesulfonamide, Valdecoxib: A Potent and Selective Inhibitor of COX-2,” J. Med. Chem. 2000, Vol. 43: 775-777, and parecoxib, sodium salt or parecoxib sodium, see, “N-[[(5-methyl-3-phenylixoxazol-4yl)-phenyl]sulfonyl]-propanimide, Sodium Salt, Parecoxib Sodium: A Potent and Selective Inhibitor of COXS-2 for Parenteral Administration,” J. Med. Chem. 2000, Vol. 43: 1661-1663. The meaning of COX-2 inhibitor in this invention also includes the substitution of the sulfonamide moiety as a suitable replacement for the methylsulfonyl moiety. See, J. Carter et al, “Synthesis and activity of sulfonamide-substituted 4,5-diaryl thiazoles as selective cyclooxygenase-2 inhibitors,” Bioorg. Med. Chem. Lett Apr. 19, 1999: Vol. 9(8): 117-174, and compounds referenced in the article “Design and synthesis of sulfonyl-substituted 4,5-diarylthiazoles as selective cyclooxygenase-2 inhibitors,” Bioorg. Med. Chem. Lett Apr. 19, 1999: Vol. 9(8): 1167-70.

In accordance with the invention, the term COX-2 inhibitor also includes NS398 as referenced in two articles: Attiga et al, “Inhibitors of Prostaglandin Synthesis Inhibit Human Prostate Tumor Cell Invasiveness and Reduce the Release of Matrix Metalloproteinases,” Cancer Research 4629-4637, Aug. 15, 2000, and in “The cyclooxygenase-2 inhibitor celecoxib induces apoptosis by blocking Akt activation in human prostate cancer cells independently of Bcl-2,” Hsu et al, 275(15) J. Biol. Chem. 11397-11403 (2000). The meaning of COX-2 inhibitor in this invention includes the cyclooxygenase-2 selective compounds referenced in Mitcheii et ai, “Cycio-oxygenase-2: pharmacology, physiology, biochemistry and relevance to NSAID therapy,” Brit. J. of Pharmacology (1999) vol.128: 1121-1132, see especially p. 1126. The meaning of COX2 inhibitor in this invention includes so-called NO—NSAIDs or nitric oxide-releasingNSAIDs referred to in L. Jackson et al, “COX-2 Selective Nonsteriodal Anti-Inflammatory Drugs: Do They Really Offer Any Advantages?” Drugs, June, 2000 vol. 59(6): 1207-1216 and the articles at footnotes 27, and 28. Also included in the meaning of COX-2 inhibitor in this invention includes any substance that selectively inhibits the COX-2 isoenzyme over the COX-1 isoenzyme in a ratio of greater than 10 to 1 and preferably in ratio of at least 40 to 1 as referenced in Winokur WO 99/20110. Also included as COX-2 inhibitors are compounds listed at page 553 in Pharmacotherapy, 4th ed: A Pathophysiologic Approach, Depiro et al (McGraw Hill 1999) including nabumetone and entodolac. Recognizing that there is overlap among the selective COX2 inhibitors set out in this paragraph, the intent of the term COX-2 inhibitor is to comprehensively include all selective COX-2 inhibitors, selective in the sense of inhibiting COX-2 over COX-1. Also included in the class of COX-2 inhibitors useful in the invention is the drug bearing the name etoricoxib referenced in the Wall Street Journal, Dec. 13, 2000 manufactured by Merck. See, also, Chauret et al, “In vitro metabolism considerations, including activity testing of metabolites, in the discovery and selection of the COX-2 inhibitor etoricoxib (MK-0663),” Bioorg. Med. Chem. Lett. 11(8): 1059-62 (Apr. 23, 2001). Another selective COX-2 inhibitor is DFU [5,5-dimethyl-3-(3-fluorophenyl)-4-(4-methylsulphonyl) phenyl-2(5H)-furanone] referenced in Yergey et al, Drug Metab. Dispos. 29(5):638-44 (May 2001).

Also included as selective COX-2 inhibitors are the flavonoid antioxidant silymarin, and an active ingredient in silymarin, silybinin, which demonstrated significant COX-2 inhibition relative to COX-1 inhibition. The silymarin also showed protection against depletion of glutathione peroxidase. Zhao et al, “Significant Inhibition by the Flavonoid Antioxidant Silymarin against 12-O-tetracecanoylphorbol 13-acetate-caused modulation of antioxidant and inflammatory enzymes, and cyclooxygenase 2 and interleukin-1 alpha expression in SENCAR mouse epidermis: implications in the prevention of stage I tumor promotion,” Mol. Carcinog. December 1999, Vol 26(4):321-33 PMID 10569809. Silymarin has been used to treat liver diseases in Europe.

The term COX-2 inhibitor includes all pharmaceutically acceptable salts for the COX-2 inhibiting compound selected. Examples of such salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamide, 2-diethylaminoethanol, 2-dimethylaminoethanol, ethanolamine, ethylenediamine, N-ethylmorpholine, Nethylpeperidine, glutamine, glucosamine, histidine, hydrabamine, isopropylamine, lysine, methyglucamine, morpholine, piperazine, piperidine, polyamine resins, procaine, purine, theobromine, triethylamine, trimethylamine, triporopylamine, troethamine, and the like.

As used herein, the term “treating” refers to preventing, alleviating, retarding, or arresting the progress of either the disorder or condition to which the term “treating” applies, including one or more symptoms of such disorder or condition. The related term “treatment,” as used herein, refers to the act of treating a disorder, symptom, or condition, as the term “treating” is defined above.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. A therapeutic change is a change in a measured biochemical characteristic in a direction expected to alleviate the disease or condition being addressed. The term “therapeutically effective amount” is also intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

As used herein, the term “bodily fluid” refers to a mixture of molecules obtained from a patient. Bodily fluids include, but are not limited to, exhaled breath, whole blood, blood plasma, urine, semen, saliva, lymph fluid, meningal fluid, amniotic fluid, glandular fluid, sputum, feces, sweat, mucous, and cerebrospinal fluid. Bodily fluid also includes experimentally separated fractions of all of the preceding solutions or mixtures containing homogenized solid material, such as feces, tissues, and biopsy samples.

The present invention provides a means for testing prostanoids production within the kidneys of subjects prone to T1D and those who have recently developed T1D to provide an assessment of those individuals at high risk for developing kidney dysfunction in the future.

One specific embodiment of the present invention provides a means for evaluating COX-2 expression and urinary and plasma prostanoid imbalances to provide a diagnostic test and to facilitate administration of treatments to prevent complications of diabetes.

In a specific embodiment, prostanoid balance is assessed by comparing prostanoid metabolites from bodily fluid sample. By way of example, prostanoid balance can be assessed by comparing urinary metabolites of renal prostanoids in persons with recent onset of T1D and pre-T1D as compared with healthy age-matched controls and Type II diabetics. An imbalance in prostanoid production is characterized by a decrease in ratio value of renal prostanoid metabolites. In a related embodiment, creatinine clearances are assessed to determine glomerular filtration and to further contribute to the assessment of risk for diabetes-related complications.

### Materials and Methods

All subjects performed a 24-hour urine collection and provided a blood sample. These samples were analyzed for: (1) 24-hour urinary prostanoid metabolites of PGE₂, PGI₂ and TXA₂ (pg/well): renal derived metabolites (PGE₂, 6 ketoPGF_{1α} (6kPGF_{1α}) and TXB₂) and systemic derived metabolites (bicyclo-PGE₂, 2,3 dinor 6 ketoPGF_{1α}, and 11-dehydro-TXB₂); (2) 24-hour urinary microalbumin levels (mg/gm Cr, normal <30); and (3) 24-hour endogenous creatinine clearance (CrCl, normal 100-120 mL/min/1.73 m²).

Analysis of ratios of PGE₂/TXB₂ and PGE₂+6keto PGF_{1α}/TXB₂ in four different groups of human subjects were performed. The four groups of subjects consisted of the following:

1) Subjects at risk for T1D (or pre-T1D) that have autoantibodies present directed toward islet antigens (ie., anti-insulin, anti islet cell, anti-GAD) but are not hyperglycemic. (n=10, 12 samples, Male:Female=6; 4, mean age=12.5 yrs).

2) Subjects with recent onset (<1 year) T1D. (n=18, 20 samples, Male:Female

=6:12, mean age=11.65).

3) Subjects with recent onset (<1 year) Type II DM. (n=7 samples, all female gender, mean age=13.43).

4) Healthy pediatric age controls, or from in pediatric renal clinic with no existing proteinuria, rental dysfunction. (n=9 samples, Male:Female=5:4, mean age=10.00).

In addition, analysis of the ratio of PGE₂/TXB₂ in two more groups of vertebrate animal subjects was performed. Non-obese diabetic (NOD) mice and BioBreeding diabetes prone (BBDP) rats were studied. The three groups of the NOD mice consisted of the following:

1) Recent onset and long-standing diabetic NOD mice (similar to human T1D). (n=20 samples, all female gender, age 12-30 weeks).

2) Pre-diabetic NOD mice (similar to human pre-T1D). (n=10 samples, all female gender, age 8-12 weeks).

3) Control non-autoimmune C57BL6 or Balb/c mice. (n=20 samples, all female gender, age matched to groups 1) and 2)).

The three groups of the BBDP rats consisted of the following:

1) Recent onset and long-standing diabetic DP rats (similar to human T1D).

2) Pre-diabetic DP rats (similar to human pre-T1D).

3) Control non-autoimmune Wistar Firth rats.

Measurement of Prostanoids

Urine was directly aliquoted and stored at −70° C. until assay. Enzyme immunoassay was used to particularly detect renal derived metabolites and the systemic derived metabolites. Measurements of urinary prostaglandin metabolites were performed on 24-hour urine samples using commercially available competitive binding enzyme immunoassay (EIA) kits (for example, BIOTRAK kits provided by AMERSHAM PHARMACIA BIOTECH AND CAYMAN CHEMICAL). The EIA kits utilize a competitive binding assay between unlabeled compound from the specimen sample and a fixed quantity of peroxidase labeled compound for a limited number of binding sites on a compound specific antibody. Assays were performed in 96 well microtitre plates with 50 μL specimen. The unknown sample was bound to appropriate antiserum and conjugate and optical density read at 450 nm using a BIORAD 3550 UV microplate reader. Raw optical density data was converted to percent binding by plotting the percent binding B/Bo as a function of the log compound concentration. Concentration of the prostanoid compound was then directly calculated in pg/well. This concentration was then converted to pg/mL and subsequently to pg/mg urinary creatinine to adjust for urinary concentration, body size and age. The final ratios of PGE₂/TXB₂ or PGE₂+6kPGF_{1α}/TXB₂ were then directly calculated. Mean values for the prostanoid concentrations/mg/Cr and prostanoid ratios were calculated directly for all the four patient groups. Plasma was separated from blood by high speed differential density centrifugation (1400 rpm, 400g×30 min) and stored at −70° C. until assay.

When assessing the prostanoid net activity and balance in an individual, measurements of urinary prostaglandins (i.e. TXB₂) require the prostanoid be extracted prior to assay.

Measurement of Creatinine

Creatinine (mg/dL) was measured using methods well understood by those skilled in the art. For example, Jaffe's assay measures creatinine levels in either serum or plasma. A modified Jaffe method may also be used (CrCl=Ucr(mg/dL)/1440]×U volume (mL)/Serum creatinine (mg/dL)). In addition, the Schwartz formula can be used to measure CrCl. The Schwartz formula is a well-established equation used to estimate CrCl in pediatric age subjects based on serum creatinine, age adjusted constant, and height (CrCl=k×H; where Cr in mg/dL; Height in cm, and where k is a constant that varies with age and gender). The urinary microalbumin levels (mg/gm Cr, normal <30) may be measured also using methods well known to the skilled artisan. For example, these levels can be measured using rate nephelometry and pyrolysis systems, such as BECKMAN SYSTEMS.

Following are examples which illustrate procedures for practicing the invention. These examples should not be construed as limiting.

### EXAMPLE 1

### Presence of Renal Hyperfiltration Prior to Hyperglycemia

As illustrated in FIG. 2, individuals with recent onset T1D (or as denoted in the figure as DPT individuals) have elevated CrCl in comparison to healthy controls. These elevations were present in both measured 24 CrCl studies as well as by Schwartz formula calculations of CrCl. These findings suggest that renal hyperfiltration is present even before the onset of hyperglycemia in children with autoantibodies directed to islet antigens. None of the subjects, however, had elevated levels of 24-hour urine microalbumin or depression in renal function. There was a weakly positive correlation of CrCl with PGE/TXB₂ (R2=0.23) and of PGE₂+PGF_{1α}/TXB₂ with CrCl (R2=0.23) but no correlation of PGE₂/Cr, TXB₂/Cr, PGF_{1α}/Cr with CrCl (R2=0.068, −0.09 and 0.05 respectively). This indicates that renal hyperfiltration is linked to prostaglandin imbalance but is not the cause of elevations in the individual levels.

### EXAMPLE 2

### Role of Renal COX-derived Prostaglandins in Renal Hyperfiltration

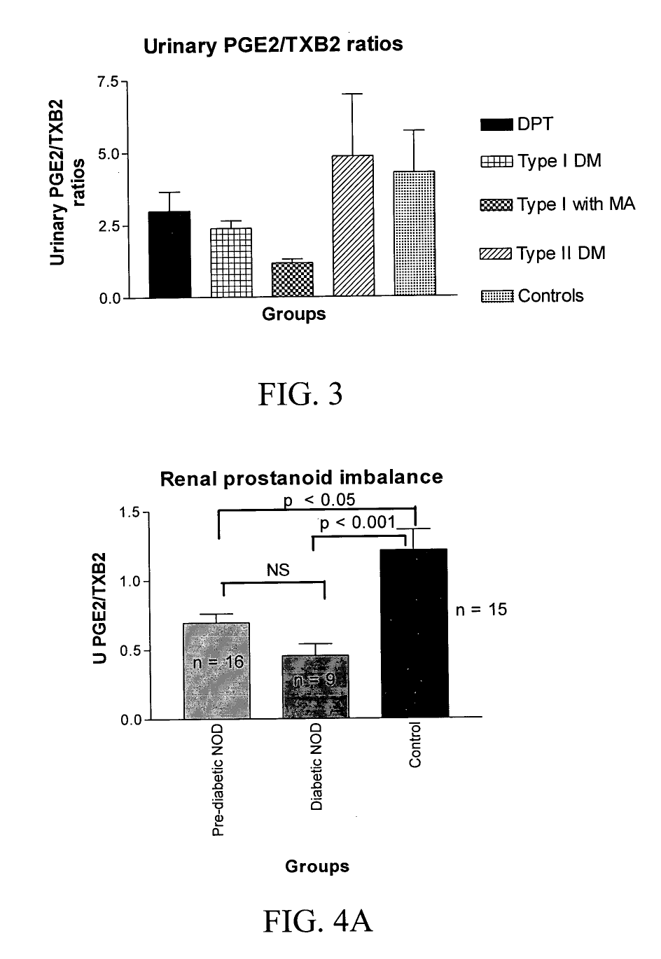

The levels of urinary metabolites of three major prostanoids (PGE₂, PGI₂, and TXA₂) known to be produced in the kidney were analyzed. 24-hr urine samples of human subjects were analyzed for their respective metabolites PGE₂, 6-ketoPGF_{1α}, and TXB₂. These metabolites represent intra-renal production rather than systemic circulating prostanoids, which are reflected by different urinary metaboites. PGE₂ and PGI₂ are known to be vasodilatory, whereas TXA₂ is a known vasoconstrictor. As shown in FIG. 3, there are alterations in the relative ratios of the prostanoids across the different subject groups. The relative balance of vasodilator to vasoconstrictor prostanoids was analyzed as a ratio to determine the relationship of these prostanoids to hyperfiltration through their hemodynamic effects. Subjects with recent onset T1D had lower urinary ratios of PGE/TXB₂ and PGE+6ketoPGF_{1α}/TXB₂ in comparison to healthy controls.

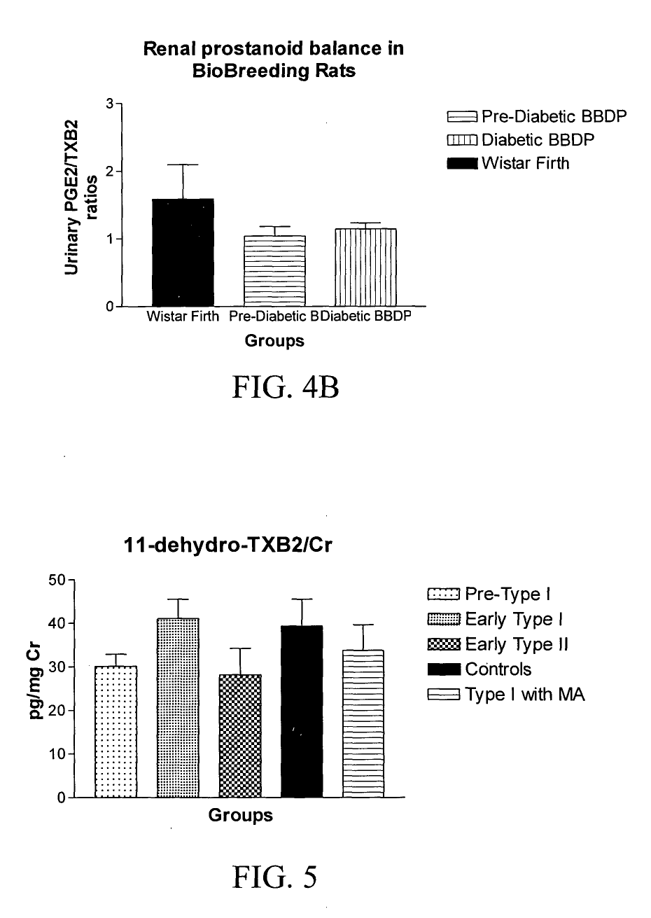

As shown in FIGS. 4a and 4b, when the same analysis was applied to pre-diabetic and diabetic NOD mice as well as pre-diabetic and diabetic DP rats, a similar shift in renal prostanoid ratios was demonstrated. The urinary PGE₂/TXB₂ ratio was significantly lower in both pre-diabetic (6-8 week old) and diabetic female (14-18 week old) NOD mice (0.69+0.06 and 0.45±0.08, respectively) in comparison to age and gender matched control B6 mice (1.21±0.14; p=0.0004 by Kruskal Wallis analysis of multiple non-parametric groups). By Dunn's post test simultaneous comparison of multiple groups, a comparison of ratios from pre-diabetic NOD mice versus control mice maintained a significant difference (p<0.05).

Further, an analysis of individual renal prostanoids, in particular PGE₂ and TXA₂ (via urinary PGE2 and TXB₂ levels) was conducted. The 24-hour urinary TXB₂ levels, adjusted for creatinine output as a ratio of TXB₂/Cr, were not different across the groups, as illustrated in FIG. 5. These data indicate that the NET prostanoid activity is better represented as the ratio of potentially opposing prostanoids rather than as levels of individual prostanoids. Thus, the balance of these substances is regulated more than the production of any one prostaglandin in the kidney.

Further, as discussed above, since excess filtration and an imbalance in prostanoids are present before full-blown T1D, high blood sugar is not the mechanism for those changes. Rather, changes in prostanoid balance and filtration are likely an inherent risk in T1D individuals that suggest future kidney problems.

In addition, urine samples from rat model of Type I DM were measured for PGE₂/TXB₂ in pre-diabetic and overtly diabetic BioBreeding Diabetes Prone (BBDP) rats. Unlike the NOD mouse, there is no gender variation in diabetes incidence in the rat model. There is a similar trend towards lower urinary PGE₂/TXB₂ ratios in the rat model as well. The urinary PGE₂/TXB₂ ratio was highest in the control WF rats (mean 15.02, S.E. 4.037, n=13), lower in the pre-diabetic BBDP (mean 10.51, S.E. 2.84, n=11) and lowest in the diabetic BBDP (mean 8.34, S.E. 1.38, n=24). Due to higher dispersions, at the current sample size, the differences are not yet statistically significant (P=0.38 by Kruskal Wallis non-parametric test of all groups simultaneously).

### EXAMPLE 3

### Role of Systemic Prostaglandins

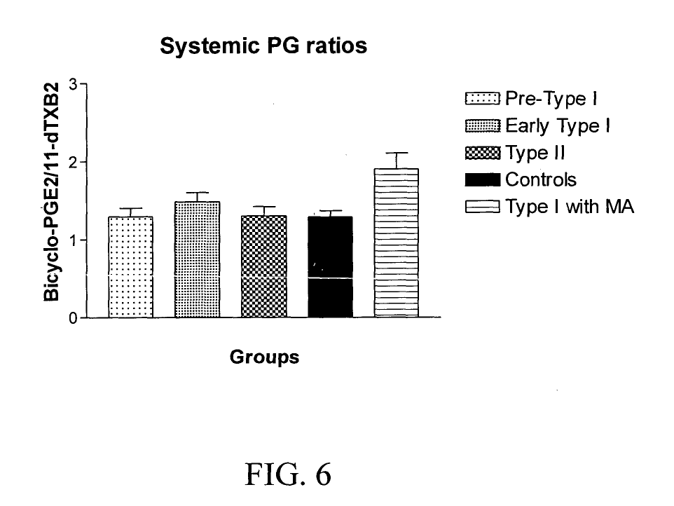

Analysis of urine samples for prostaglandin metabolites was conducted to assess the production and levels of systemic circulating prostaglandins (bicyclo-PGE₂ for PGE₂; 11-dehydroTXB₂ for TXA₂). Bicyclo-PGE₂ and 11-dehydroTXB₂ were analyzed to determine if systemically circulating prostaglandins play a role in renal hyperfiltration, either due to filtration of excessive levels of systemic prostaglandins or due to local renal effects of high systemic ratios. The ratio of urinary bicyclo-PGE₂ to 11-dehydroTxB₂ for TXA₂ was not significantly different across groups, as illustrated in FIG. 6.

Further, as illustrated in FIG. 5, examination of absolute levels of the prostanoid metabolites for systemic PGE₂ and TXA₂ demonstrated no significant differences in urinary 11-dehydroTXB₂ levels in T1D or bicyclo-PGE₂ to 11-dehydroTXB₂ ratios between the groups.

### EXAMPLE 4

### COX-2 Expression

This example demonstrates the role of COX-2 enzyme dysregulation in producing alterations in renal prostaglandin balance. Selective COX-2 and COX-1 blockage were analyzed in three groups of mice—NOD mice, B6 mice, and a congenic B6.NODc1t strain.

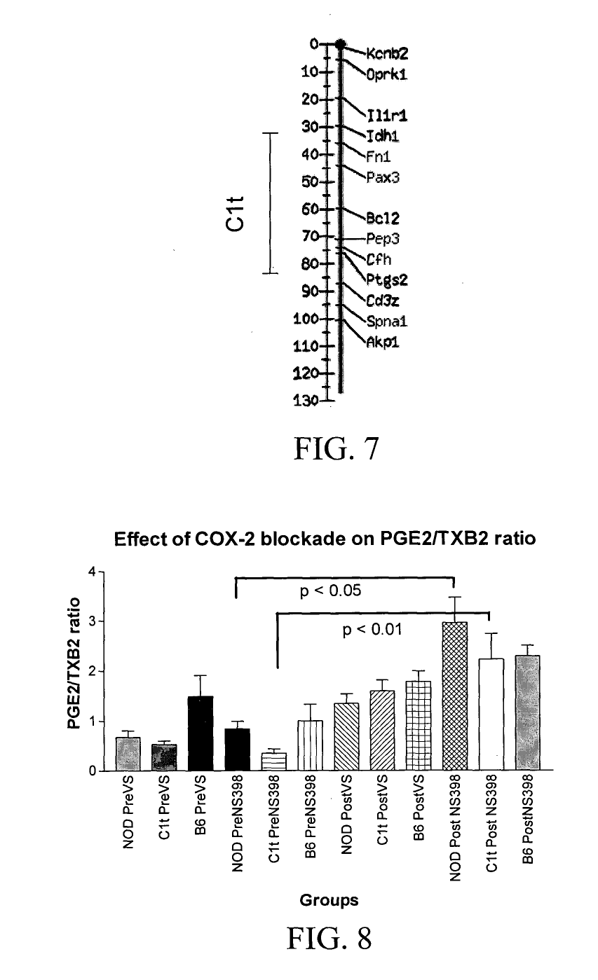

The B6.NODc1t strain mouse develops insulitis but does not develop diabetes mellitus. The B6.NODc1t strain mouse contains an interval of chromosome 1 from the NOD mouse, on a B6 genetic background. As illustrated in FIG. 7, the C1t interval size is 47cM and the centromeric and stelomeric end markers are D1MIT5 and D1MIT15, respectively. The COX-2 gene in mice is present in the C1t interval of chromosome 1, thus allowing for assessment of the behavior of the COX-gene independent of the effects of other diabetes susceptibility genes on other chromosomes.

Eight age-matched female 6-8 week old mice of each strain (NOD, B6, and C1t) were divided into two groups. Group A received a selective COX-2 blocker NS-398 10 mg/kg/d i.p. daily for 14 days, while Group B received a selective COX-1 blocker (valeryl salicylic acid 30 mg/kg/d i.p. daily) for the same length of time. Urinary ratios of PGE₂/TXB₂ were measured on timed urine collections pre- and post-intervention and were significantly different across the groups (P=0.0069 by Kruskal Wallis non-parametric test of all groups).

As shown in FIG. 8, the COX-2 inhibitor NS-398 lead to a 3-7 fold increase in the urinary PGE₂/TXB₂ ratio in all the three strains of mice. At baseline, the NOD and B6.NODc1t mice had lower urinary ratios of PGE₂/TXB₂ in comparison to B6 mice. After completion of the intervention, the ratio was almost identical in all three strains, indicating a greater effect in the NOD and B6.NODc1t strains. The difference in urinary PGE₂/TXB₂ was significant for the NOD and B6.NODc1t group that received NS-298 by Dunn's simultaneous multiple comparison test. There was a small non-significant elevation in urinary PGE₂/TXB₂ in the group that received the selective COX-1 inhibitor valeryl salicylic acid.

### EXAMPLE 5

### Time of COX-2 Expression in Pre-Diabetic Stage NOD Kidney

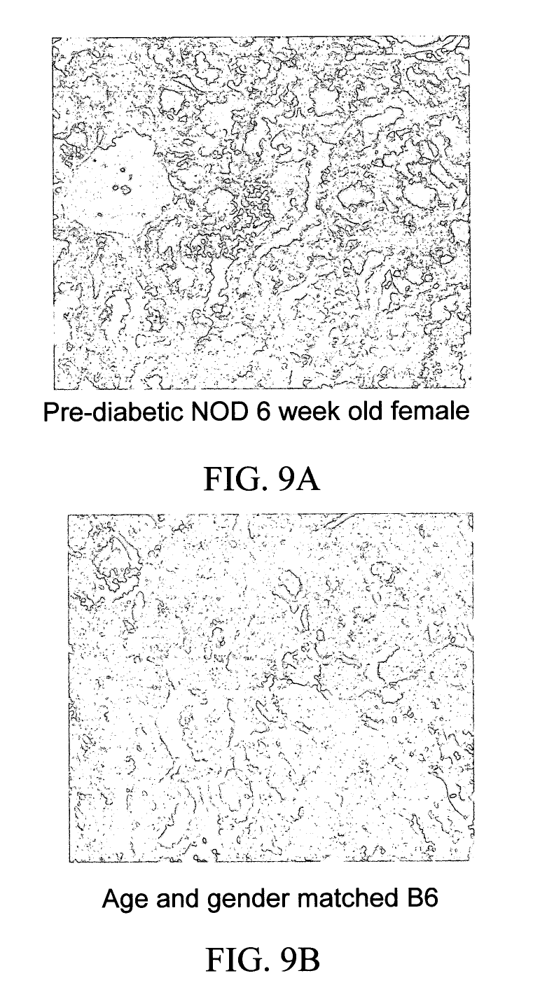

Kidney tissue was harvested from age-matched pre-diabetic 6-8 week old female NOD and B6 mice. The tissue was fixed in a zinc-based fixative and embedded in a wax medium that preserved superior tissue staining characteristics compared to formalin/paraffin. The primary antibody used was a polyclonal antibody to murine COX2 (Cayman Chemical; 1:50 dilution), bound to secondary peroxidase labeled anti-rat IgG and revealed with DAB. As shown in FIGS. 9a and 9b, pre-diabetic 6-8 week old NOD mice showed specific COX-2 staining within proximal convoluted tubular cells adjacent to cortical glomeruli. Age-matched control B6 mice did not exhibit similar staining. These data show that COX-2 expression occurs in the kidneys of diabetes susceptible animals at early time points, prior to hyperglycemia, at a time when autoimmunity and insulitis are already established.

### EXAMPLE 6

### Uses, Formulations, and Administration

To restore prostanoid balance and prevent or delay the development of diabetes-related complications, selective COX-2 inhibitors or other compounds that modulate eicosanoid production can be administered prior to or at the onset of T1D. Application of the treatments of the subject invention can be accomplished by any suitable method and technique presently or prospectively known to those skilled in the art.

In one embodiment, selective COX-2 inhibitors are effective in restoring prostanoid balance. Pharmaceutical compositions containing selective COX-2 inhibitors as active ingredients are useful in treating pre-T1D or recent onset T1D individuals to prevent or delay the development of diabetes-related complications.

The dosage administered will be dependent upon the response desired; the type of host involved; its age, health, weight, kind of concurrent treatment, if any; frequency of treatment; therapeutic ration and like considerations.

According to the present invention, the compounds that restore prostanoid balance and prevent/delay the development of diabetes-related complications can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

In accordance with the invention, pharmaceutical compositions comprising an active ingredient and one or more non-toxic, pharmaceutically acceptable carrier or diluent.

The compositions of the invention are advantageously used in a variety of forms, i.e., tablets, capsules, pills, powders, aerosols, granules, and oral solutions or suspensions and the like containing suitable quantities of the active ingredient. Such compositions are referred to herein and in the accompanying claims generically as “pharmaceutical compositions.” Typically, they can be in unit dosage form, namely, in physically discrete units suitable as unitary dosages for human or animal subjects, each unit containing a predetermined quantity of active ingredient calculated to produce the desired therapeutic or prophylactic effect in association with one or more pharmaceutically acceptable other ingredients, i.e., diluent or carrier.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for identifying individuals at risk for developing diabetes-related complications comprising assaying at least one bodily fluid sample from the individual for at least two different prostanoids, calculating at least one ratio of the prostanoids as an indicator of prostanoid levels, detecting an imbalance in prostanoid levels using the ratio of the prostanoids, wherein the imbalance indicates a likelihood of developing diabetes-related complications.

2. The method, according to claim 1, wherein the imbalance in prostanoid levels is characterized by a decrease in the ratio of the prostanoids in the individual as compared to a normal net ratio of prostanoids in the individual.

3. The method, according to claim 1, wherein the imbalance in prostanoid levels is characterized by a decrease in the ratio of the prostanoids in the individual as compared to a normal net ratio of prostanoids in at least one other normal, healthy individual.

4. The method, according to claim 1, wherein the imbalance in prostanoid levels is characterized by a decrease in the ratio of the prostanoids in the individual as compared to a normal net ratio of prostanoids in at least one other individual diagnosed with recent onset Type II diabetes.

5. The method, according to claim 1, wherein the prostanoids are selected from the group consisting of: PGE₂, PGI₂, TXA₂, TXB₂, 6 ketoPGF_{1α}, bicyclo-PGE₂, 2,3 dinor 6 ketoPGF_{1α}, and 11-dehydro-TXB₂.

6. The method, according to claim 1, wherein the prostanoids are urinary prostanoid metabolites of PGE₂, PGI₂, and TXA₂.

7. The method, according to claim 1, wherein the ratio of prostanoids is PGE₂/TXB₂.

8. The method, according to claim 1, wherein the ratio of prostanoids is PGE₂+6kPGF_{1α}/TXB₂.

9. The method, according to claim 1, further comprising the step of assessing creatine clearances.

10. The method, according to claim 1, further comprising the step of assessing cyclooxygenase-2 expression.

11. The method, according to claim 1, wherein the diabetes-related complications are selected from the group consisting of: neuropathy, nephropathy, cardiomyopathy, myocardial infarction, ophthalmopathy, retinopathy, and atherosclerosis.

12. A method for decreasing or preventing the development of diabetes-related complications comprising restoring balanced prostanoid levels by administering a therapeutically effective amount of an eicosanoid modulator to the individual.

13. The method, according to claim 12, further comprising the steps of: assaying from an individual's bodily fluid sample at least two different prostanoids, calculating at least one ratio of the prostanoids as an indicator of prostanoid levels, and detecting an imbalance in prostanoid levels using the ratio of the prostanoids, wherein the imbalance indicates a likelihood of developing diabetes-related complications.

14. The method, according to claim 12, wherein the prostanoids are selected from the group consisting of: PGE₂, PGI₂, TXA₂, TXB₂, 6 ketoPGF_{1α}, bicyclo-PGE₂, 2,3 dinor 6 ketoPGF_{1α}, and 11-dehydro-TXB₂.

15. The method, according to claim 12, wherein the prostanoids are urinary prostanoid metabolites of PGE₂, PGI₂, and TXA₂.

16. The method, according to claim 12, wherein the ratio of prostanoids is PGE₂/TXB₂.

17. The method, according to claim 12, wherein the ratio of prostanoids is PGE₂+6kPGF_{1α}/TXB₂.

18. The method, according to claim 12, further comprising the step of assessing creatine clearances.

19. The method, according to claim 12, further comprising the step of assessing cyclooxygenase-2 expression.

20. The method, according to claim 12, wherein the eicosanoid modulator is a cyclooxygenase inhibitor.

21. The method, according to claim 20, wherein the cyclooxygenase-2 inhibitor is selected from the group consisting of: rofecoxib, celecoxib, valdecoxib, parecoxib, and NS398.

22. The method, according to claim 12, wherein the diabetes-related complications are selected from the group consisting of: neuropathy, nephropathy, cardiomyopathy, myocardial infarction, ophthalmopathy, retinopathy, and atherosclerosis
